(19) United States
(12) Patent Application Publication
(10) Pub. No.: US 2024/0245528 A1
(43) Pub. Date: Jul. 25, 2024

(54) TOOL FOR PLACEMENT OF AN ACETABULAR CUP

(71) Applicant: OSTIUM GROUP, SAINT ETIENNE DE MONTLUC (FR)

(72) Inventors: Edgard SOQUENNE, SAINT ETIENNE DE MONTLUC (FR); Vincent RETAILLEAU, SAINT ETIENNE DE MONTLUC (FR); Léopold BIBARD, SAINT ETIENNE DE MONTLUC (FR); Corentin LE HENANF, SAINT ETIENNE DE MONTLUC (FR)

(21) Appl. No.: 18/424,725

(22) Filed: Jan. 26, 2024

Related U.S. Application Data
(63) Continuation of application No. PCT/EP2022/071268, filed on Jul. 28, 2022.

(30) Foreign Application Priority Data
Jul. 28, 2021 (FR) 2108216

(51) Int. Cl. A61F 2/46

(52) U.S. Cl. CPC A61F 2/4609

(57) ABSTRACT

A longilineal member, which may serve as a handle, and may be formed of a synthetic or other relatively soft material, is designed to engage the threaded hole of an acetabular cup, for the manipulation of the cup during surgery. A force distribution element, shaped to conform to the inner curved surface of the cup is trapped between the cup and the longilineal member so as to reduce the forces on the threaded portion of the longilineal member during such manipulations, so as to avert the risk of the thread stripping.

### FIELD OF THE INVENTION

The present invention relates to instruments for surgery of the hip, and in parmodular a tool for placement of an acetabular cup is provided.

### BACKGROUND OF THE INVENTION

The human hip joint bears a substantial part of an individual's weight, yet is subject to sustained movement over a significant angular range. The joint can deteriorate over an individual's lifetime, and this may be exacerbated by other medical conditions. The extension of average life expectancy over the twentieth century has further increased the likelihood that a given individual will experience hip problems at some stage. Since the 1950s, surgical procedures have been developed for the replacement of an organic hip joint with a mechanically equivalent mechanism fabricated from suitable engineering materials.

FIG. 1 shows a typical hip joint replacement mechanism.

In accordance with the relevant surgical procedure, the pelvic acetabulum is excavated using a hemispherical reamer instrument to form a substantially hemispherical cavity suitably sized to receive the cup 110, into which the cup is inserted and secured by means of suitable retaining means, for example screws screwed through holes in the cup into the patient's pelvis, bone cement, etc. The cup may be formed of stainless steel, titanium, or other suitably hard, durable and anatomically inert materials. The outer surface of the cup may be provided with surface features intended to mechanically engage the user's pelvis, and/or to surface textures or treatments designed to promote osseointegration such as cellular scaffold structures and the like. The cup itself has a coaxial, broadly hemispherical cavity, which is typically shaped to receive a liner 120. This liner is typically formed of a durable, low friction material, providing the internal bearing of the new hip joint. Common materials include polythene or ceramics. This liner may then receive a prosthetic ball head 130, in place of the head of the patient's femur. The prosthetic ball head 130 is connected to the patient's femur by a neck 140 secured by a femoral stem (not shown), inserted into the femur. During the surgical procedure to insert a mechanism such as that shown in FIG. 1, the cup must be maneuvered into position through a narrow surgical incision, and carefully aligned with respect to the patient's anatomy so as to avoid a modified or unbalanced gait or posture after completion of the procedure. Once positioned, the cup must be fully inserted so as to fully engage the walls of the cavity. This may require considerable force, without compromising control.

FIG. 2a shows the acetabular cup of FIG. 1, without a liner.

As shown in FIG. 2a, the acetabular cup 110 comprises a threaded hole 111 at its base, during the insertion procedure, the threaded hole may conveniently be used as a means for the manipulation of the acetabular cup, specifically by engaging the threaded hole with a correspondingly threaded rod 200, which may be formed for convenient positioning of the cup during surgery. A steel or titanium rod 200 screwed into a steel or titanium cup will provide a suitably strong and rigid joint, enabling the surgeon to apply considerable force to the cup via the rod.

FIG. 2b shows the acetabular cup of FIG. 2a engaged with a positioning rod. Once the cup is in position, the rod may be rotated to disengage from the cup, leaving the cup in position.

Traditional surgical instruments such as the rod 200 are formed of stainless steel. Stainless steel can be sterilised by means of an autoclave or the like. However, the large number of individual instruments used in certain surgical interventions, and total hip replacement in particular, as well as the growing number of patients requiring such surgery, can lead to a significant logistical burden. Meanwhile, the increasing incidence of antibiotic resistant bacteria makes full control of the sterilisation cycle even more important. In this context, the use of single use, recyclable instruments is highly desirable made of plastics or the like is highly desirable. A drawback of plastic materials is that they are less hard and durable than steel or titanium—if rod 200 were formed of plastic; the threads would soon be stripped by the harder metal of the acetabular cup.

EP2561835 discloses an orthopedic impactor device for positioning an orthopedic prosthetic cup implant (56) during a hip replacement surgery is described. The impactor is designed with a “one piece” elongated body, having a distal prosthetic cup engagement portion which is separated from a proximal strike plate by an elongated body and handle portions. A connection rod, connectable to an orthopedic prosthetic cup, is positioned longitudinally within the through-bores of the body and handle portions. A lever arm, having a linkage member, is pivotally connected to the rod such that when the lever arm is pivoted towards the exterior surface of the annular sidewall of the handle portion, the distal end of the rod moves proximally within the body portion.

US20090192515 presents an acetabular inserter aids a surgeon in controlling the installation of an acetabular cup prosthesis having a central, female aperture. The inserter includes a head, a housing and a locking mechanism. The housing is attached to the head, the housing enclosing a drive train having, at a far end, a prosthesis engaging thread, and at the opposite end, a handle which facilitates turning of the drive train by the operator. The locking mechanism is associated with the housing which selectively locks the drive train, and thus the prosthesis, in position. The opposite end of the drive train has a latch device which enables quick removal from the housing for cleaning and sterilization.

US20130204264 presents various embodiments of components, devices, systems and methods are provided for a pneumatic surgical instrument having a probe or an impactor disposed at a distal end thereof and configured to make contact with a selected portion of an orthopedic implant or device and drive the implant into a hole or void formed in a patient's bone. The instrument is configured to generate a shock wave, which is then transferred to the distal end of the probe or impactor, and thence into the orthopedic implant, thereby causing the implant to be driven into contact with portions of the void or hole.

It is desirable to provide an instrument for positioning an acetabular cup which is compatible with manufacture from a softer material than typically used for the fabrication of surgical instruments.

### SUMMARY OF THE INVENTION

In accordance with the present invention in a first aspect there is provided a tool for placement of an acetabular cup, the tool comprising a longilineal member formed of a thermoplastic or other synthetic material and providing a thread section at a distal extremity thereof for engaging an acetabular cup, the tool further comprising a force distribution element formed of a thermoplastic or other synthetic material, the force distribution element defining a surface shaped to conform to an inner surface of the acetabular cup, and comprising a central cavity shaped to receive a distal portion of the longilineal member, such that when the longilineal member is inserted through the force distribution element, the thread part of the cylindrical portions protrudes to an extent necessary to engage the acetabular cup, and whereby the acetabular cup engaging the thread locks the longilineal member, the force distribution element and the acetabular cup in a respectively fixed relationship with regard to forces lateral to the longilineal member.

In a development of the first aspect, the force distribution element is substantially tear-drop shaped.

In a development of the first aspect, the longilineal member has a circular cross section and is further provided with a locking element to prevent free rotation of the force distribution element in the central cavity, the central cavity being keyed correspondingly to allow insertion of the longilineal member and the locking element to prevent rotation thereof once inserted

In a development of the first aspect, the force distribution element and the longilineal member are respectively configured to engage in a bayonet fashion, such that rotation of the longilineal member with respect to the force distribution element in one direction about the axis of the longilineal member locks the two together, and rotation in the other frees the two so that they may be separated.

In a development of the first aspect, the force distribution element and the longilineal member are respectively configured to engage in a bayonet fashion, such that rotation of the longilineal member with respect to the force distribution element in either direction locks the two together, with a central position allowing separation.

In a development of the first aspect, the force distribution element and the longilineal member are provided with resilient retention structures so that a minimum force is required to engage and disengage one element from the other.

In a development of the first aspect, the force distribution element and the longilineal member are provided with resilient elements configured to bias the longilineal member away from the release position.

In a development of the first aspect, a substantial length of the longilineal member is also threaded, to engage corresponding helical structures on the inner surface of the central cavity of the force distribution element.

In a development of the first aspect, the threads engaging the force distribution element have the same direction as the acetabular cup.

In a development of the first aspect, the threads engaging the force distribution element may have the opposite direction to those of the acetabular cup.

In a development of the first aspect, the force distribution element comprises a plurality of lateral indentations.

In a development of the first aspect, the force distribution element comprises a plurality of vertical lumens, extending from the upper surface of the force distribution element. In a development of the first aspect, one or more of the vertical lumens extend from the upper surface of the force distribution element through to the distal surface of the force distribution element.

In a development of the first aspect, the longilineal member or force distribution element is composed of a synthetic material or a synthetic composite material.

In a development of the first aspect, the longilineal member or force distribution element holder is composed of a glass fibre reinforced polyarylamide.

### BRIEF DESCRIPTION OF THE DRAWINGS

The above and other advantages of the present invention will now be described with reference to the accompanying drawings, for illustration purposes only, in which: FIG. 1 shows a typical hip joint replacement mechanism;

FIG. 2a shows the acetabular cup of FIG. 1, without a liner;

FIG. 2b shows the acetabular cup of FIG. 2a engaged with a positioning rod; FIG. 3 shows a tool for placement of an acetabular cup in a first embodiment;

FIG. 4 shows a cross section of the longilineal member and distribution element highlighting locking configurations according to certain embodiments;

FIG. 5 shows a tool for placement of an acetabular cup in a further embodiment;

FIG. 6 shows a first variant in the form of the force distribution element;

FIG. 7 shows a second variant in the form of the force distribution element;

FIG. 8a shows a partially disassembled modular tool in accordance with an embodiment; and

FIG. 8b shows an assembled modular tool in accordance with an embodiment.

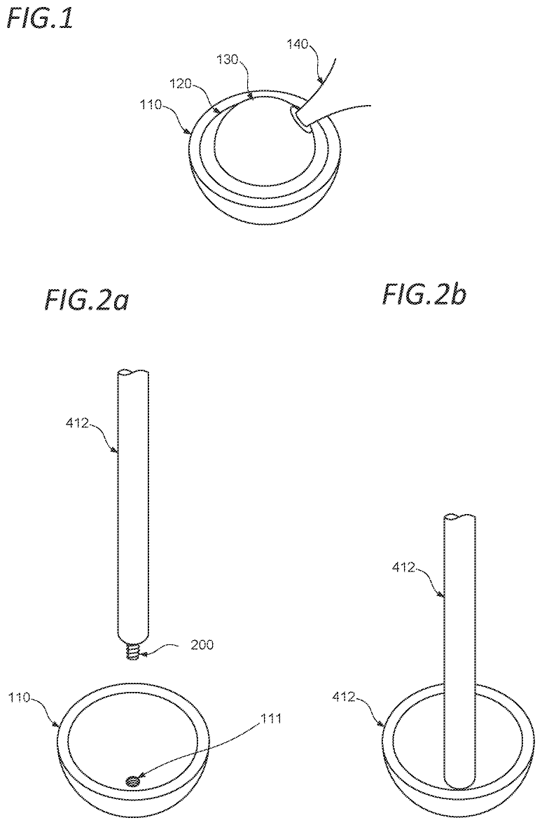

### DETAILED DESCRIPTION

FIG. 3 shows a tool for placement of an acetabular cup in a first embodiment.

As shown, the tool 300 comprises a longilineal member 350 formed of a thermoplastic or other synthetic material and providing a threaded section at a distal extremity 351 thereof.

A distal extremity of the cylindrical section 352 is provided with a thread 353 for engaging an acetabular cup 310.

The tool 300 further comprises a force distribution element 360 formed of a thermoplastic or other synthetic material. The force distribution element 360 defines a surface shaped to conform to an inner surface 313 of the acetabular cup 310, and comprises a central cavity shaped to receive a distal portion of the longilineal member, such that when the a longilineal member is inserted through the force distribution element, the thread part of the cylindrical portion protrudes to an extent necessary to engage the acetabular cup.

Accordingly, when the acetabular cup engages the thread, the longilineal member, force distribution element and acetabular cup are locked in a respectively fixed relationship with regard to forces lateral to the longilineal member. As shown in the embodiment of FIG. 3, this is achieved by means of a shoulder 351 where the longilineal member expands radially beyond the dimensions of the central cavity 361 of the force distribution element 360 so that when the threaded portion is screwed into an acetabular cup, the shoulder 351 abuts a proximal extremity of the force distribution element 360, and progressively press the force distribution element 360 against the inner surface 313 of the acetabular cup 310. With the acetabular cup 310, longilineal member and force distribution element 360 thus disposed, the three elements are substantially solidary, such that a surgeon may firmly manipulate the acetabular cup 310 through an exertion on force on a proximal extremity of the longilineal member.

It will be appreciated that the prior art approach of FIG. 1 uses the threaded hole of the acetabular cup for the dual purposes of fixing it to the threaded rod 200, and also as a load bearing surface for transmitting lateral, axial and torsional forces to the acetabular cup. The present invention separates these functions, and retains the use of the threaded hole for the less demanding role of fixing the longilineal member to the acetabular cup, whilst spreading the lateral, axial and torsional forces during surgery across the broad interface area between the longilineal member and the inner and proximal outer walls of the force distribution element 360 on one hand, and between the broad interface area between the acetabular cup and the distal outer walls of the force distribution element 360 on the other.

The longilineal member may be circular in cross section (cylindrical), or may have any other form as may be convenient, for example square or hexagonal.

A non circular cross section may be advantageous in that when the longilineal member is twisted to unscrew it from the acetabular cup, a rotation will also be imparted to the force distribution element 360, which may tend to facilitate its disengagement from the acetabular cup.

As shown, the force distribution element 360 is substantially tear-drop shaped, with a rounded distal portion shaped to conform to the inner surface of the acetabular cup, and slowed shoulders extending outward from the proximal extremity to meet the outer edge of the rounded distal portion, so as to provide an effective transmission of forces from the longilineal member to the acetabular cup. It will be appreciated that while this form may be suggested by the sole consideration of force distribution, other factors such as ease of manipulation, economy of materials or weight may suggest variations on this form, for example as discussed further below.

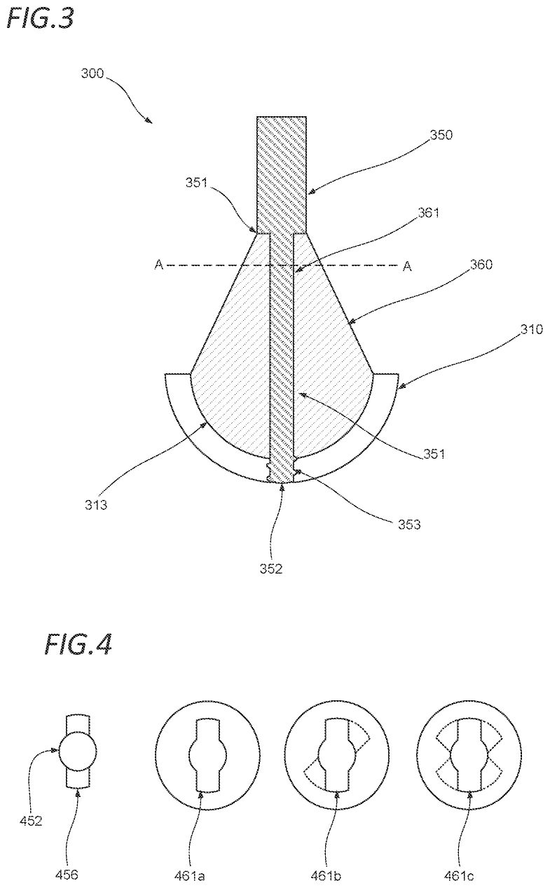

FIG. 4 shows a cross section of the longilineal member and distribution element highlighting locking configurations according to certain embodiments.

By way of example, FIG. 4 presents a cross section corresponding to section A-A of FIG. 3 according to certain variants.

In certain embodiments, the longilineal member and distribution element 360 may also be provided with locking elements so as to control the degrees of freedom of movement of one with respect to the other.

In particular, while it is intended that it be possible to insert the longilineal member into the distribution element 360, the two should not be free to disengage inadvertently, especially within the body of the patient.

To this end, where the longilineal member has a circular cross section 452 (i.e. is cylindrical or tapered towards its distal extremity), it may also be provided with a locking element 456 such as a locking bar to prevent free rotation of the force distribution element 360 about the longilineal member. On this basis, the central cavity 461a as illustrated may be correspondingly keyed. Still further, the force distribution element 360 and/or longilineal member may be configured to engage in a bayonet fashion, such that rotation of the longilineal member with respect to the force distribution element 360 in one direction locks the two together, and rotation in the other frees the two so that they may be separated. This may be achieved by providing suitable formations within the central cavity 461b as illustrated. Still further, the force distribution element 360 and/or longilineal member may be configured to engage in a bayonet fashion, such that rotation of the longilineal member with respect to the force distribution element 360 in either direction direction locks the two together, with a central position allowing separation. This may be achieved by providing suitable formations within the central cavity 461c as illustrated.

Still further, the longilineal member and force distribution element 360 may be provided with suitable resilient retention structures such as a spring loaded ball plunger in one element engaging a detent in the other way so that a minimum force is required to engage and disengage one element from the other, and/or resilient elements configured to bias the longilineal member away from the release position.

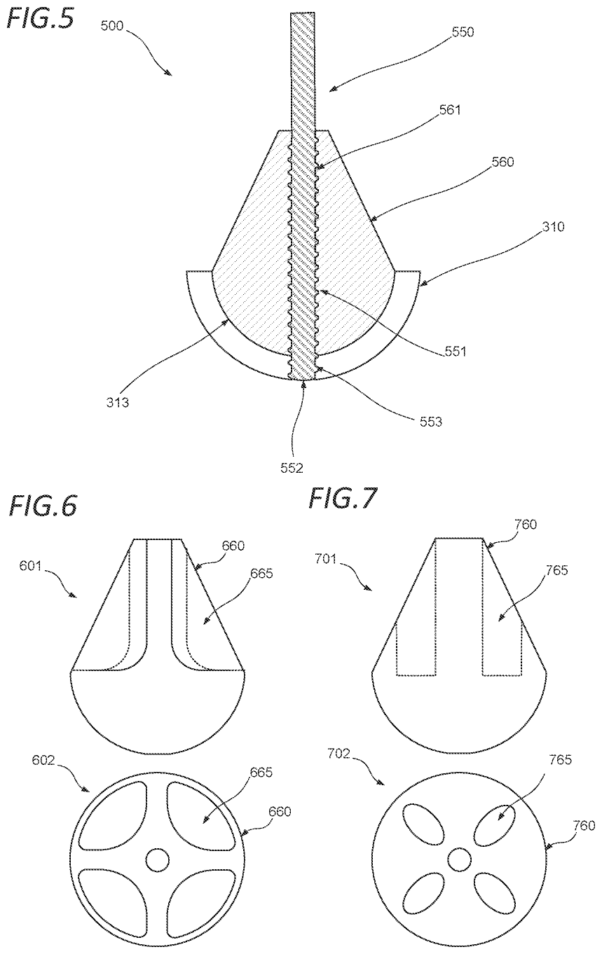

FIG. 5 shows a tool for placement of an acetabular cup in a further embodiment.

As shown, the tool 500 comprises a longilineal member 550 formed of a thermoplastic or other synthetic material and providing a threaded section at a distal extremity 551 thereof substantially as described with reference to FIG. 3.

As shown in FIG. 5 meanwhile, not only is the distal extremity of the cylindrical section 552 is provided with a thread for engaging an acetabular cup 310, but in this embodiment a substantial length of the longilineal member 551 is also threaded, to engage corresponding helical structures on the inner surface of the central cavity 561 of the force distribution element 560.

On this basis, in this embodiment the force distribution element 560 must be screwed onto the longilineal member 551 along at least part of its length. The threads engaging the force distribution element 560 may have the same direction (left handed or right handed) as the acetabular cup 310. This has the benefit that the surgeon may perform the same gesture when mounting the acetabular cup 310 and the force distribution element 560 of the longilineal member 551.

The threads engaging the force distribution element 560 may have the opposite direction (left handed or right handed) to those of the acetabular cup 310. This has the benefit that once the acetabular cup 310 is in position in the patient, twisting the longilineal member 551 in the appropriate direction to unscrew the longilineal member 551 from the acetabular cup 310 will not at the same time unscrew the longilineal member 551 from the longilineal member 551.

While FIGS. 3 and 5 show the force distribution element 560 as solid blocks, other forms may be desirable in the interests of reducing weight and the consumption of raw materials.

FIG. 6 shows a first variant in the form of the force distribution element.

FIG. 6 presents a first angle projection view of a first variant in the form of the force distribution element.

Element 601 is a side view of the force distribution element 660. Element 602 is a plan view of the force distribution element 660.

As shown, the force distribution element 660 comprises four lateral indentations 665, which may also be seen as describing fins or buttresses extending substantially from the circular plane corresponding to the top of the acetabular cup, to the vertical column surrounding the central cavity as described above. These buttresses accordingly serve to transmit forces from the acetabular cup, to the vertical column surrounding the central cavity, with a reduced use of material, and correspondingly a reduced weight.

An additional benefit of this structure is that the fins provide for an easy grip when manipulating and particularly screwing or unscrewing the force distribution element. It will be appreciated that while FIG. 6 shows fourfold symmetry, any number of fins may be provided, e.g. 3, 5, 6, 7, 8 or more fins (and correspondingly, lateral indentations).

Furthermore, these need not necessarily be regularly spaced.

FIG. 7 shows a second variant in the form of the force distribution element.

FIG. 7 presents a first angle projection view of a second variant in the form of the force distribution element.

Element 701 is a side view of the force distribution element 760. Element 702 is a plan view of the force distribution element 760.

As shown, the force distribution element 660 comprises four vertical lumens 765, extending from the upper surface of the force distribution element. As shown, these lumens terming substantially in the circular plane corresponding to the top of the acetabular cup. These lumens accordingly a reduced use of material, and correspondingly a reduced weight, whilst leaving the distribution of forces throughout the element substantially unchanged.

Although the lumens are shown as substantially cylindrical, they may take any convenient form.

It will be appreciated that while FIG. 7 shows fourfold symmetry, any number of lumens may be provided, e.g. 3, 5, 6, 7, 8 or more lumens. Furthermore, these need not necessarily be regularly spaced, either axially or radially.

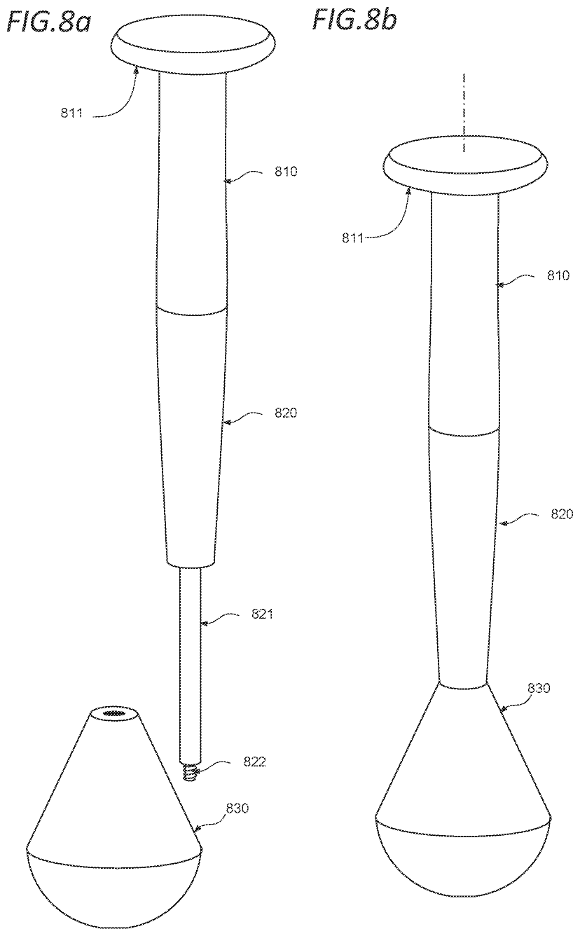

Furthermore, some or all of the lumens may extend through to the distal surface of the force distribution element which in use abuts the inner surface of the acetabular cup. This may provide an additional benefit of permitting the flow of air or fluids so as to break a vacuum between the force distribution element and the inner surface of the acetabular cup which may otherwise prevent easy separation of the elements in use. FIG. 8a shows a partially disassembled modular tool in accordance with an embodiment.

As shown, there is provided a modular tool a handle 810, a tool holder 820 and a force distribution element 860. The handle 910 may comprises a releasable coupling allowing the tool holder 820 to be releasably connected thereto, or the handle and tool holder may form a unitary component. A distal extremity of the tool holder 820 presents a longilineal member 821, substantially as described above adapted to be slidingly inserted into a force distribution element 830 substantially as described above, and provided with a thread 822 as described previously.

FIG. 8b shows an assembled modular tool in accordance with an embodiment. FIG. 8b shows the tool of FIG. 8a in a fully assembled configuration.

As shown, there is provided a modular tool a handle 810, a tool holder 820 and a force distribution element 860. The handle 910 may comprises a releasable coupling allowing the tool holder 820 to be releasably connected thereto, or the handle and tool holder may form a unitary component. A distal extremity of the tool holder 820 presents a longilineal member 821, substantially as described above slidingly inserted into the force distribution element 830 substantially as described above, and provided with a thread 822 as described previously.

The handle may optionally be provided with an angle datum such as a radial line on the guard plate, or a radial lumen through which a bar may by inserted.

As shown, the handle further comprises an optional guard plate 811 at the proximal end thereof. Such a guard plate may serve to protect the hand of a user when gripping the handle 920 from blows struck against the proximal end thereof with a hammer, mallet or the like, for example where the tool or working part 960 is a chisel, reamer or other such tool requiring a percussive application.

The force distribution element, and indeed any longilineal member, tool holder or handle, of any embodiment may be formed a thermoplastic or other synthetic material. It may in particular be formed from a polyamide, for example a polyarylamide. The synthetic material may comprise additional components such as a filler, swelling agent and the like. It may still further be formed of a synthetic composite material, comprising a glass, carbon fibre, carbon nanoparticle or any other material exhibiting a high tensile strength, in a matrix of a synthetic material, such as any of those listed above. In certain embodiments, the tool holder may be composed of a glass fibre reinforced polyarylamide, such as for example that marketed by the Solvay corporation under the trademark “Ixef GS 1022”.

Force distribution element, and indeed any longilineal member, tool holder or handle, of any embodiments may be formed of different materials in different regions, including metal parts and synthetic parts. The handle, tool holder or longilineal member may also comprise voids for the purpose of economy of material, reduced weight and so on.

Where the force distribution element, is incorporated in a modular tool as shown in FIG. 8, the tool holder 820 and/or longilineal member and/or handle 810 may each be composed of the materials mentioned above. In some embodiments, the handle, tool holder and working part may all be composed of the same material.

A tool as described herein may be useful both for the trial fitting of acetabular cups, and for their definitive installation, as well as for their eventual removal.

As such, there is provided a set of modular elements which is calculated to provide the elements required for a planned surgical intervention, taking into account the specifics of the patient, in particular their physical dimensions. In particular, a series of progressively sized acetabular cups may be provided, with respectively different force distribution elements, corresponding to the inner surface of the cups. A single longilineal element may be provided, compatible will all of the force distribution elements, or in a case where some or all of the acetabular cups have holes of different dimensions or thread specifications, multiple longilineal elements may be provided accordingly.

Additionally, the modular parts are pre-associated taking into account the planned sequence of planned surgical steps in the scheduled intervention. This reduces the number of manipulations in adapting modular tools during the operation procedure.

By this means, a minimal set of tools may be provided for a particular intervention, which together with the multiple applications of certain elements of the modular tool system reduces the weight and amount of used material to a minimum.

Accordingly, throughout the foregoing embodiments there is provided a longilineal member, which may serve as a handle, and may be formed of a synthetic or other relatively soft material, is designed to engage the threaded hole of an acetabular cup, for the manipulation of the cup during surgery. A force distribution element, shaped to conform to the inner curved surface of the cup is trapped between the cup and the longilineal member so as to reduce the forces on the threaded portion of the longilineal member during such manipulations, so as to avert the risk of the thread stripping.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A tool for placement of an acetabular cup, said tool comprising a longilineal member formed of a thermoplastic or other synthetic material and providing a thread section at a distal extremity thereof for engaging an acetabular cup, said tool further comprising a force distribution element formed of a thermoplastic or other synthetic material, said force distribution element defining a surface shaped to conform to an inner surface of said acetabular cup, and comprising a central cavity shaped to receive a distal portion of said longilineal member, such that when said longilineal member is slidingly inserted through said force distribution element, said thread part protrudes to an extent necessary to engage said acetabular cup, and whereby said acetabular cup engaging said thread locks said longilineal member, said force distribution element and said acetabular cup in a solidary and respectively fixed relationship with regard to forces lateral to said longilineal member.

2. The tool of claim 1 wherein said force distribution element is substantially tear-drop shaped.

3. The tool of claim 1 wherein said longilineal member has a circular cross section and is further provided with a locking element to prevent free rotation of the force distribution element in said central cavity, said central cavity being keyed correspondingly to allow insertion of said longilineal member and said locking element to prevent rotation thereof once inserted.

4. The tool of claim 3 wherein said force distribution element and said longilineal member are respectively configured to engage in a bayonet fashion, such that rotation of the longilineal member with respect to the force distribution element in one direction about the axis of said longilineal member locks the two together, and rotation in the other frees the two so that they may be separated.

5. The tool of claim 3 wherein said force distribution element and said longilineal member are respectively configured to engage in a bayonet fashion, such that rotation of the longilineal member with respect to the force distribution element in either direction locks the two together, with a central position allowing separation.

6. The tool of claim 3, wherein said force distribution element and said longilineal member are provided with resilient retention structures so that a minimum force is required to engage and disengage one element from the other.

7. The tool of claim 1 wherein said force distribution element comprises a plurality of vertical lumens, extending from the upper surface of the force distribution element.

8. The tool of claim 7 wherein one or more of said vertical lumens, extend from the upper surface of the force distribution element through to the distal surface of the force distribution element.

9. The tool of claim 1 wherein said longilineal member or said force distribution element is composed of a synthetic composite material.

10. The tool of claim 9 wherein said longilineal member or said force distribution element is composed of a glass fibre reinforced polyarylamide.
